(19) United States
(12) Patent Application Publication
(10) Pub. No.: US 2019/0337868 A1
(43) Pub. Date: Nov. 7, 2019

(54) METHOD OF PREPARING METHANE USING GAMMA-VALEROLACTONE

(71) Applicant: Dongguan University of Technology, Dongguan (CN)

(72) Inventors: Shimin KANG, Dongguan (CN); Yong WANG, Dongguan (CN); Zhanfu GU, Dongguan (CN); Yongjun XU, Dongguan (CN); Jingwen ZHOU, Dongguan (CN); Taijie LI, Dongguan (CN)

(21) Appl. No.: 16/144,858

(22) Filed: Sep. 27, 2018

(30) Foreign Application Priority Data
May 3, 2018 (CN) 201810416449.4

(51) Int. Cl. C07C 1/207; C07C 9/04; B01J 31/20

(52) U.S. Cl. CPC C07C 1/2076; C07C 9/04; C07C 2531/20; B01J 2531/821; B01J 31/20

(57) ABSTRACT

The present invention relates a method of preparing methane using γ-valerolactone. A solution of γ-valerolactone is mixed with a triruthenium dodecacarbonyl catalyst, for a reaction at 150° C.-250° C. for 1 to 12 hours, and then subjected to cooling; wherein a mass ratio of γ-valerolactone to the triruthenium dodecacarbonyl catalyst is between 1:2 and 1:50; and the solution of γ-valerolactone has a mass concentration of 50 g/L-300 g/L. In the present invention, γ-valerolactone is converted into methane rapidly by a one-step catalysis deoxygenation using a triruthenium dodecacarbonyl catalyst. The preparation method provided by the present invention can realize a complete conversion of γ-valerolactone, and the methane gas has a yield up to 45 wt %. Besides, such method has characteristics of short reaction time, high yield of methane, easy collection, simple process and convenient operation, and it has industrialized application prospect.

### TECHNICAL FIELD

The present invention relates to a technical field of preparation of renewable gas fuel, and particularly relates to a method of preparing methane using gamma-valerolactone (“γ-valerolactone”).

### BACKGROUND

As the increasing consumption of resources of petroleum and natural gas, development and application of renewable alternative energy are of great significance. Biomass resource is a renewable, and a large amount of biology-based compounds, such as γ-valerolactone, can be prepared from the biomass resource. γ-valerolactone can be prepared on a large scale by an acid hydrolysis of lignocellulosic biomass and then a hydrogenation reaction.

Development of utilization approaches of γ-valerolactone is a problem to be solved necessarily, and at present stage γ-valerolactone can be used as a solvent, an intermediate of organic synthesis and etc. Besides, application of γ-valerolactone in a fossil-alternative fuel is a significant topic. One γ-valerolactone contains two oxygen atoms and presents in a form of lactone, and thus γ-valerolactone has a high water-solubility and a low heat value which greatly limit its application as fuel. A possible solution to solve this problem is to seek an efficient and environmental catalysis technology, which converts γ-valerolactone into a low-molecular weight hydrocarbon fuel by deoxygenation, so as to substitute the fossil fuel.

Methane is a hydrocarbon having the least carbon content and the most hydrogen content, and it is widely used for civil use and industries, for example used as natural gas and coal gas, and also used as an original material to produce important chemicals such as ethyne, carbon black, dichloromethane, tetrachloromethane and etc. As exhaustion of the resources of petroleum and natural gas, methane will become a significant energy. At present, methane can be prepared by microbiological fermentation and obtained by decomposing the organic materials in a biogas digester. However, such process requires a long time and a high demand of control accuracy in process conditions (such as pH value and temperature). Also, the preparation of methane by fermentation of microorganism may at the same time generate gas which is harmful to the environment, such as hydrogen sulfide. Therefore, it is of great significance to prepare methane by a technology which is simple, efficient, environmental and fast.

### SUMMARY OF THE INVENTION

An objective of the present invention is to overcome the deficiency in the prior art, that is, to provide a method of preparing methane using γ-valerolactone. In the present invention, γ-valerolactone is converted into methane rapidly by a one-step catalysis deoxygenation using a triruthenium dodecacarbonyl catalyst. This method can realize a complete conversion of γ-valerolactone, and the methane gas has a yield up to 45 wt %.

In order to realize the above objective, the present invention adopts the following technical solution:

A method of preparing methane using γ-valerolactone, a solution of γ-valerolactone is mixed with a triruthenium dodecacarbonyl catalyst, for a reaction at 150° C.-250° C. for 1 to 12 hours, and then subjected to cooling.

In the present invention, a gas product may be obtained after the reaction is completed and cooled to room temperature. The gas product may be collected in a gas tank directly through a discharge valve of a reaction kettle, and components and yield of the gas product may be determined by a componential analysis method for refinery gas. Carbon dioxide is the main non-hydrocarbon product in said gas product, while methane is the main hydrocarbon product in the gas, in addition to a small amount of ethane, propane, butane and pentane.

In the present invention, γ-valerolactone is converted into methane gas by one-step deoxygenation under a hydrothermal condition, solving the problem that application of γ-valerolactone as a renewable fuel is limited owing to containing oxygen element. The preparation method of methane provided by the present invention has characteristics of short reaction time (1 to 12 hours), high yield of methane, easy collection of the gas product, catalyst being capable of being reused, being environmentally friendly, simple process and convenient operation.

In the present invention, the catalyst may be reused without separation. During the continuous reaction, it only requires maintaining a reaction temperature to be constant and adding γ-valerolactone continuously for reaction. The gas product obtained after the reaction is completed and cooled to room temperature may be collected in the gas tank directly through the discharge valve of the reaction kettle, and thereby repeating this process.

Preferably, a reaction temperature is 180° C.-240° C., and a reaction time is 3 to 12 hours.

Preferably, a mass ratio of γ-valerolactone to the triruthenium dodecacarbonyl catalyst is between 1:2 and 1:50.

Preferably, the mass ratio of γ-valerolactone to the triruthenium dodecacarbonyl catalyst is between 1:2 and 1:20.

Preferably, the solution of γ-valerolactone has a mass concentration of 50 g/L-300 g/L.

Preferably, the solution of γ-valerolactone has a mass concentration of 50 g/L-150 g/L.

Preferably, the reaction is carried out under stirring with a stirring rate of 100 rpm-300 rpm.

Preferably, temperature is increased to 150° C.-250° C. at a heating rate of 5° C./min-10° C./min.

Compared with the prior art, the present invention has following beneficial effects:

The preparation method provided by the present invention converts γ-valerolactone completely into methane. During the reaction, oxygen in the γ-valerolactone is removed in a form of carbon dioxide, without generating other harmful gas and with the yield of methane up to 45 wt %. This solves the problem that application of γ-valerolactone as a renewable fuel is limited owing to containing oxygen element. In this method, triruthenium dodecacarbonyl serves as the catalyst which has high catalytic activity and is capable of being reused, under a gentle reaction condition for a short reaction time, while γ-valerolactone can be converted by 100%. Additionally, the gas product prepared by the method provided by the present invention can be collected easily, and the process is simple with convenient operation, having great promotion and application value.

### BRIEF DESCRIPTION OF THE DRAWINGS

FIG. 1 shows a GC-FID analysis diagram of a gas product in embodiment 1.

### DETAILED DESCRIPTION OF THE PREFERRED EMBODIMENT

The present invention is further described as follows in combination with specific embodiments and accompanied drawings, but the embodiments do not limit the present invention in any way. Unless specified, reagents, methods and apparatus used in the present invention are conventional reagents, methods and apparatus in the art.

Unless specified, reagents and materials used in the present invention are commercially available.

### Embodiment 1

In the embodiment, γ-valerolactone was catalyzed and converted to prepare methane by using triruthenium dodecacarbonyl, and specific steps are as follows:

(1) 4 g of a triruthenium dodecacarbonyl catalyst and 100 mL of a 80 g/L γ-valerolactone solution were added to a 300 mL high-temperature high-pressure reaction kettle. Mechanical stirring was turned on, with a stirring rate controlled at 300 rpm. Temperature was programmed and increased to 200° C. at a heating rate of 10° C./min, kept at 200° C. for 12 hours, and cooled to room temperature with cold water after the reaction was completed.

Gas product obtained was collected in a gas tank and analyzed by a componential analysis method for refinery gas. A yield of the obtained methane was 45 wt %. Besides, ethane with a yield of 1 wt %, propane with a yield of 0.3 wt % and butane with a yield of 0.3 wt % were obtained. HPLC (high performance liquid chromatography) analysis of an aqueous solution in the reaction kettle showed that γ-valerolactone was converted completely.

(2) 8 g of γ-valerolactone was added to the aqueous solution which was after the reaction of step (1), and a concentration of γ-valerolactone was kept at 80 g/L. The reaction kettle was sealed and the mechanical stirring was turned on, with the stirring rate controlled at 200 rpm. Temperature was programmed and increased to 200° C. at a heating rate of 10° C./min, kept at 200° C. for 12 hours, and cooled to room temperature with cold water after the reaction was completed.

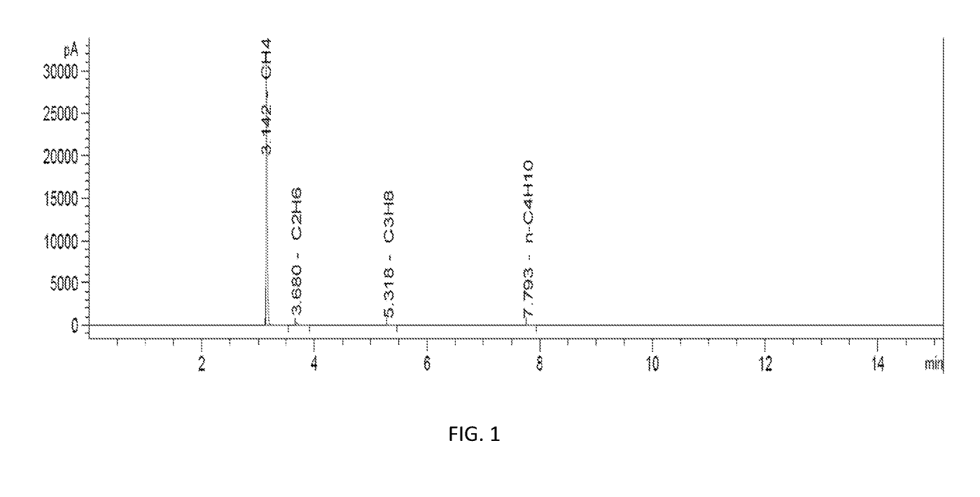

Gas product obtained was collected in the gas tank and analyzed by GC-FID (a diagram is shown as FIG. 1). The yield of the obtained methane was 43 wt %. HPLC analysis of the aqueous solution in the reaction kettle showed that γ-valerolactone was converted completely.

(3) 8 g of γ-valerolactone was added to the aqueous solution which was after the reaction of step (2), and the concentration of γ-valerolactone was kept at 80 g/L. The reaction kettle was sealed and the mechanical stirring was turned on, with the stirring rate controlled at 200 rpm. Temperature was programmed and increased to 200° C. at a heating rate of 10° C./min, kept at 200° C. for 12 hours, and cooled to room temperature with cold water after the reaction was completed.

Gas product obtained was collected in the gas tank and analyzed by the componential analysis method for refinery gas. The yield of the obtained methane was 40 wt %. HPLC analysis of the aqueous solution in the reaction kettle showed that γ-valerolactone was converted completely.

(4) γ-valerolactone was added to the aqueous solution which was after the reaction of step (3), and the concentration of γ-valerolactone was kept at 80 g/L. The reaction kettle was sealed and the mechanical stirring was turned on, with the stirring rate controlled at 200 rpm. Temperature was programmed and increased to 200° C. at a heating rate of 10° C./min and kept at 200° C. for 12 hours. After the reaction was completed, the yield of the obtained methane was 34 wt %.

(5) γ-valerolactone was added to the aqueous solution which was after the reaction of step (4), and the concentration of γ-valerolactone was kept at 80 g/L. The reaction kettle was sealed and the mechanical stirring was turned on, with the stirring rate controlled at 200 rpm. Temperature was programmed and increased to 200° C. at a heating rate of 10° C./min and kept at 200° C. for 12 hours. After the reaction was completed, the yield of the obtained methane was 24 wt %.

### Embodiment 2

In the embodiment, γ-valerolactone was catalyzed and converted to prepare methane by using triruthenium dodecacarbonyl, and specific steps are as follows:

(1) 1 g of the triruthenium dodecacarbonyl catalyst and 100 mL of a 100 g/L γ-valerolactone solution were added to a 300 mL high-temperature high-pressure reaction kettle. Mechanical stirring was turned on, with a stirring rate controlled at 200 rpm. Temperature was programmed and increased to 240° C. at a heating rate of 5° C./min, kept at 240° C. for 10 hours, and cooled to room temperature with cold water after the reaction was completed.

Gas product obtained was collected in a gas tank and analyzed by the componential analysis method for refinery gas. A yield of the obtained methane was 29 wt %. Besides, ethane with a yield of 0.5 wt %, propane with a yield of 0.1 wt % and butane with a yield of 0.1 wt % were obtained. HPLC analysis of an aqueous solution in the reaction kettle showed that γ-valerolactone was converted completely.

(2) 10 g of γ-valerolactone was added to the aqueous solution which was after the reaction of step (1), and a concentration of γ-valerolactone was kept at 100 g/L. The reaction kettle was sealed and the mechanical stirring was turned on, with the stirring rate controlled at 200 rpm. Temperature was programmed and increased to 240° C. at a heating rate of 5° C./min, kept at 240° C. for 10 hours, and cooled to room temperature with cold water after the reaction was completed. Gas product obtained was collected in the gas tank and analyzed by the componential analysis method for refinery gas. The yield of the obtained methane was 31 wt %.

(3) γ-valerolactone was added to the aqueous solution which was after the reaction of step (2), and the concentration of γ-valerolactone was kept at 100 g/L. The reaction kettle was sealed and the mechanical stirring was turned on, with the stirring rate controlled at 200 rpm. Temperature was programmed and increased to 240° C. at a heating rate of 5° C./min, kept at 240° C. for 10 hours, and cooled to room temperature with cold water after the reaction was completed. Gas product obtained was collected in the gas tank and analyzed by the componential analysis method for refinery gas. The yield of the obtained methane was 19 wt %.

### Embodiment 3

In the embodiment, γ-valerolactone was catalyzed and converted to prepare methane by using triruthenium dodecacarbonyl, and specific steps are as follows:

1 g of the triruthenium dodecacarbonyl catalyst and 250 mL of a 200 g/L γ-valerolactone solution were added to a 300 mL high-temperature high-pressure reaction kettle. Mechanical stirring was turned on, with a stirring rate controlled at 300 rpm. Temperature was programmed and increased to 250° C. at a heating rate of 6° C./min, kept at 250° C. for 12 hours, and cooled to room temperature with cold water after the reaction was completed. Gas in which a main product was methane was obtained.

### Embodiment 4

In the embodiment, γ-valerolactone was catalyzed and converted to prepare methane by using triruthenium dodecacarbonyl, and specific steps are as follows:

2 g of the triruthenium dodecacarbonyl catalyst and 100 mL of a 40 g/L γ-valerolactone solution were added to a 300 mL high-temperature high-pressure reaction kettle. Mechanical stirring was turned on, with a stirring rate controlled at 300 rpm. Temperature was programmed and increased to 150° C. at a heating rate of 8° C./min, kept at 150° C. for 12 hours, and cooled to room temperature with cold water after the reaction was completed. Gas in which a main product was methane was obtained.

### Embodiment 5

In the embodiment, γ-valerolactone was catalyzed and converted to prepare methane by using triruthenium dodecacarbonyl, and specific steps are as follows:

10 g of the triruthenium dodecacarbonyl catalyst and 100 mL of a 300 g/L γ-valerolactone solution were added to a 300 mL high-temperature high-pressure reaction kettle. Mechanical stirring was turned on, with a stirring rate controlled at 100 rpm. Temperature was programmed and increased to 220° C. at a heating rate of 8° C./min, kept at 220° C. for 3 hours, and cooled to room temperature with cold water after the reaction was completed. Gas in which a main product was methane was obtained.

### Embodiment 6

In the embodiment, γ-valerolactone was catalyzed and converted to prepare methane by using triruthenium dodecacarbonyl, and specific steps are as follows:

5 g of the triruthenium dodecacarbonyl catalyst and 100 mL of a 100 g/L γ-valerolactone solution were added to a 300 mL high-temperature high-pressure reaction kettle. Mechanical stirring was turned on, with a stirring rate controlled at 100 rpm. Temperature was programmed and increased to 250° C. at a heating rate of 8° C./min, kept at 250° C. for 1 hour, and cooled to room temperature with cold water after the reaction was completed. Gas in which a main product was methane was obtained.

### Embodiment 7

In the embodiment, γ-valerolactone was catalyzed and converted to prepare methane by using triruthenium dodecacarbonyl, and specific steps are as follows:

5 g of the triruthenium dodecacarbonyl catalyst and 100 mL of a 200 g/L γ-valerolactone solution were added to a 300 mL high-temperature high-pressure reaction kettle. Mechanical stirring was turned on, with a stirring rate controlled at 200 rpm. Temperature was programmed and increased to 180° C. at a heating rate of 8° C./min, kept at 180° C. for 3 hours, and cooled to room temperature with cold water after the reaction was completed. Gas in which a main product was methane was obtained.

### Comparative Embodiment 1

Except for without adding the triruthenium dodecacarbonyl catalyst, other steps of the preparation method of the present comparative embodiment are as same as those of Embodiment 3. A yield of the obtained methane in the present comparative embodiment was only 2%. Compared with that which was added with the catalyst, the yield of methane was too low.

### Comparative Embodiment 2

Except for a reaction temperature at 120° C., other steps of the preparation method of the present comparative embodiment are as same as those of Embodiment 3. A conversion rate of γ-valerolactone in the present comparative embodiment was less than 5%. The yield of the obtained methane was negligible.

### Comparative Embodiment 3

Except for a reaction temperature at 280° C., other steps of the preparation method of the present comparative embodiment are as same as those of Embodiment 3. The triruthenium dodecacarbonyl catalyst was decomposed under a condition of 280° C. in the present comparative embodiment. A catalytic efficiency was low and the catalyst cannot be reused.

### Comparative Embodiment 4

Except for a reaction time for 0.5 hour, other steps of the preparation method of the present comparative embodiment are as same as those of Embodiment 3. γ-valerolactone failed to be converted substantially in the present comparative embodiment, and the yield of the obtained methane was negligible.

Objectives, technical solutions and beneficial effects of the present invention are further described by the above specific implementations. It should be understood that the above description is merely specific implementation of the present invention, and does not limit the scope of protection of the present invention. All modifications, equivalent substitution and improvement within the spirit and the principle of the present invention shall be included in the scope of protection of the present invention.

What is claimed is:

1. A method of preparing methane using γ-valerolactone, characterized in that, a solution of γ-valerolactone is mixed with a triruthenium dodecacarbonyl catalyst, for a reaction at 150° C.-250° C. for 1 to 12 hours, and then subjected to cooling.

2. The method according to claim 1, wherein a reaction temperature is 180° C.-240° C., and a reaction time is 3 to 12 hours.

3. The method according to claim 1, wherein a mass ratio of γ-valerolactone to the triruthenium dodecacarbonyl catalyst is between 1:2 and 1:50.

4. The method according to claim 3, wherein the mass ratio of γ-valerolactone to the triruthenium dodecacarbonyl catalyst is between 1:2 and 1:20.

5. The method according to claim 1, wherein the solution of γ-valerolactone has a mass concentration of 50 g/L-300 g/L.

6. The method according to claim 5, wherein the solution of γ-valerolactone has a mass concentration of 80 g/L-150 g/L.

7. The method according to claim 1, wherein the reaction is carried out under stirring with a stirring rate of 100 rpm-300 rpm.

8. The method according to claim 1, wherein temperature is increased to 150° C.-250° C. at a heating rate of 5° C./min-10° C./min.
